(19) United States
(12) Patent Application Publication
(10) Pub. No.: US 2026/0118193 A1
(43) Pub. Date: Apr. 30, 2026

(54) DETECTION DEVICE

(71) Applicant: Japan Display Inc., Tokyo (JP)

(72) Inventor: Ayato KITAMURA, Tokyo (JP)

(21) Appl. No.: 19/431,377

(22) Filed: Dec. 23, 2025

Related U.S. Application Data
(63) Continuation of application No. PCT/JP2024/019489, filed on May 28, 2024.

(30) Foreign Application Priority Data
Jun. 29, 2023 (JP) 2023-107160

(51) Int. Cl. G01L 1/24; A61B 5/02; A61B 5/1455; A61B 90/00; G06V 40/145; G08B 21/18

(52) U.S. Cl. CPC G01L 1/248; A61B 5/02007; A61B 90/06; G06V 40/145; G08B 21/182; A61B 5/14552; A61B 2090/064; A61B 2562/0238

(57) ABSTRACT

According to an aspect, a detection device includes: a sensor comprising a plurality of optical sensor elements arranged two-dimensionally; and an acquirer configured to acquire a pattern of a blood vessel of a human finger that is included in a light intensity pattern of light detected by the sensor. The acquirer is configured to acquire information on force from the finger toward the sensor based on the light intensity pattern.

### CROSS-REFERENCE TO RELATED APPLICATION

This application claims the benefit of priority from Japanese Patent Application No. 2023-107160 filed on Jun. 29, 2023 and International Patent Application No. PCT/JP2024/019489 filed on May 28, 2024, the entire contents of which are incorporated herein by reference.

### BACKGROUND

### 1. Technical Field

What is disclosed herein relates to a detection device.

### 2. Description of the Related Art

Widely known are systems that detect light by an optical sensor with a human finger pressed against the optical sensor, thereby detecting blood vessels in the finger (e.g., Japanese Patent Application Laid-open Publication No. 2004-265269).

It is known that, in the detection of blood vessels in a finger, an image of the blood vessels blurs if the force from the finger against the optical sensor is too strong. Therefore, it is desirable to detect the state in which the force from the finger against the optical sensor is too strong, and provide feedback to a user who is pressing the finger against the optical sensor. To provide such feedback using a conventional configuration, however, it is necessary to provide a dedicated force sensor to detect the force from the finger against the optical sensor, resulting in an increase in the number of parts and cost.

For the foregoing reasons there is a need for a detection device that can acquire information on force from a finger without a dedicated force sensor.

### SUMMARY

According to an aspect, a detection device includes: a sensor comprising a plurality of optical sensor elements arranged two-dimensionally; and an acquirer configured to acquire a pattern of a blood vessel of a human finger that is included in a light intensity pattern of light detected by the sensor. The acquirer is configured to acquire information on force from the finger toward the sensor based on the light intensity pattern.

### BRIEF DESCRIPTION OF THE DRAWINGS

FIG. 1 is a block diagram illustrating a main configuration example of a detection device;

FIG. 2 is a schematic illustrating the positional relation between a sensor module, a light source part, and the configuration around them, and a blood vessel of a finger serving as an object to be detected by the sensor module;

FIG. 3 is a plan view illustrating an example of the sensor module, the light source part, and the configuration coupled to them;

FIG. 4 is a block diagram illustrating a more detailed functional configuration example of the configuration illustrated in FIG. 3;

FIG. 5 is a circuit diagram illustrating the sensor module;

FIG. 6 is a circuit diagram illustrating a plurality of partial detection areas;

FIG. 7 is a schematic illustrating the relation between the degree of force applied from the finger to a sensor and the results of sensing by the sensor;

FIG. 8 is a schematic illustrating a case where a change in the area occupied by a dark region in the sensing results corresponds to movement;

FIG. 9 is a schematic illustrating a case where a change in the area occupied by the dark region in the sensing results corresponds to expansion;

FIG. 10 is a flowchart illustrating the procedure performed by the detection device, including determination and notification that the force from the finger toward the sensor is too strong;

FIG. 11 is a block diagram illustrating a main configuration example of the detection device according to a modification; and

FIG. 12 is a schematic illustrating the positional relation between the sensor module, the light source part, an operating part, and the configuration around them in the detection device according to the modification, and the blood vessel of the finger serving as the object to be detected by the sensor module.

### DETAILED DESCRIPTION

The following describes an embodiment of the present disclosure with reference to the drawings. What is disclosed herein is merely an example, and the present disclosure naturally encompasses appropriate modifications easily conceivable by those skilled in the art while maintaining the gist of the present invention. To further clarify the description, the drawings may schematically illustrate, for example, widths, thicknesses, and shapes of various parts as compared with actual aspects thereof. However, they are merely examples, and interpretation of the present disclosure is not limited thereto. The same element as that illustrated in a drawing that has already been discussed is denoted by the same reference numeral through the description and the drawings, and detailed description thereof may not be repeated where appropriate.

### Embodiments

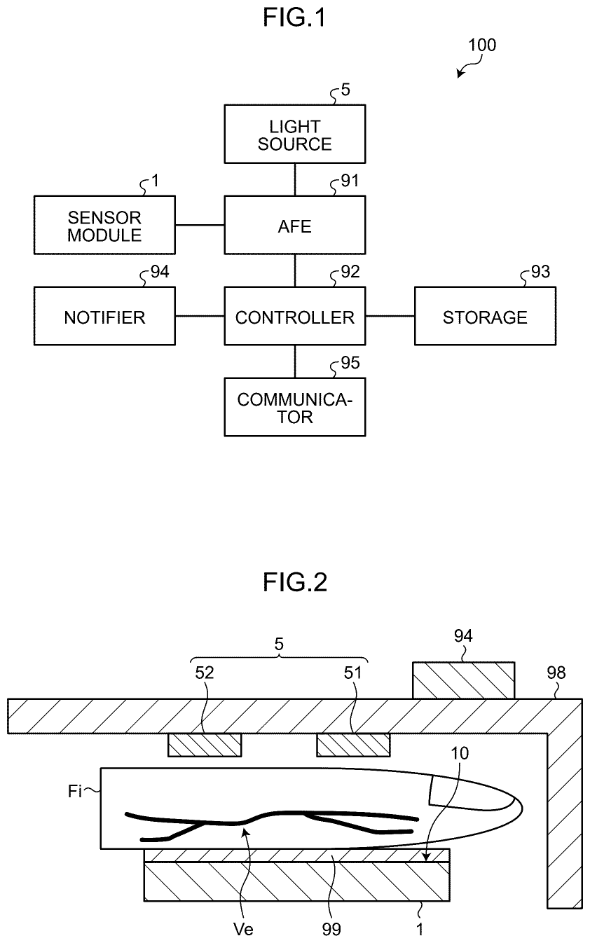

FIG. 1 is a block diagram illustrating a main configuration example of a detection device 100 according to an embodiment. The detection device 100 includes a sensor module 1, a light source part 5, an AFE 91, a controller 92, a storage 93, a notifier 94, and a communicator 95. The sensor module 1 is provided to be able to detect light. The light source part 5 is a light source that emits light with wavelengths detectable by the sensor module 1. The AFE with the reference numeral 91 refers to an analog front-end.

FIG. 2 is a schematic illustrating the positional relation between the sensor module 1, the light source part 5, and the configuration around them, and a blood vessel Ve of a finger Fi serving as an object to be detected by the sensor module 1. As illustrated in FIG. 2, the light source part 5 includes, for example, a first light source base member 51 and a second light source base member 52. The specific configuration example of the light source part 5 illustrated in FIG. 2 is given by way of example only, and the configuration is not limited thereto. For example, the light source part 5 may include one of the first light source base member 51 and the second light source base member 52 and a light source provided to the one of them. Alternatively, the light source part 5 may include three or more light source base members and light sources provided to the light source base members.

A sensor 10 of the sensor module 1 faces the light source part 5 with the finger Fi interposed therebetween. While light from the light sources (e.g., a first light source 61 and a second light source 62, which will be described later) provided in the light source part 5 is partially blocked and absorbed by the finger Fi and the blood vessel Ve in the finger Fi, the light not blocked or absorbed is detected by photodiodes PD provided to the sensor 10. The blood vessel Ve and the part of the finger Fi other than the blood vessel Ve have a difference in the degree of blocking and absorption of light. The difference creates a contrast between the blood vessel Ve and the part of the finger Fi other than the blood vessel Ve. The detection device 100 can detect the pattern of the blood vessel Ve by causing the sensor module 1 to detect the light and visualizing the contrast.

In the example illustrated in FIG. 2, a louver 99 is provided between the sensor module 1 and the finger Fi. The louver 99 is, for example, a light-shielding member having a plurality of light guide holes passing therethrough in the direction in which the finger Fi and the sensor 10 face, and acts to limit the direction of travel of light passing between the finger Fi and the sensor 10 to the direction in which the finger Fi and the sensor 10 face.

In the example illustrated in FIG. 2, the light source part 5 is supported by a light-shielding member 98 on the side opposite the sensor 10 with the finger Fi interposed therebetween. The light-shielding member 98 is a cover-like member that covers the detection surface of the sensor 10 to prevent light other than the light emitted from the light source part 5 from being incident on the sensor 10. The light source part 5 is provided on the surface facing the sensor 10 (one surface) of the light-shielding member 98. In the example illustrated in FIG. 2, the notifier 94 is provided to the light-shielding member 98. The notifier 94 is provided on the other surface of the light-shielding member 98. The notifier 94 appears to be provided on the light-shielding member 98 by a user who puts the finger Fi between the light source part 5 and the sensor 10.

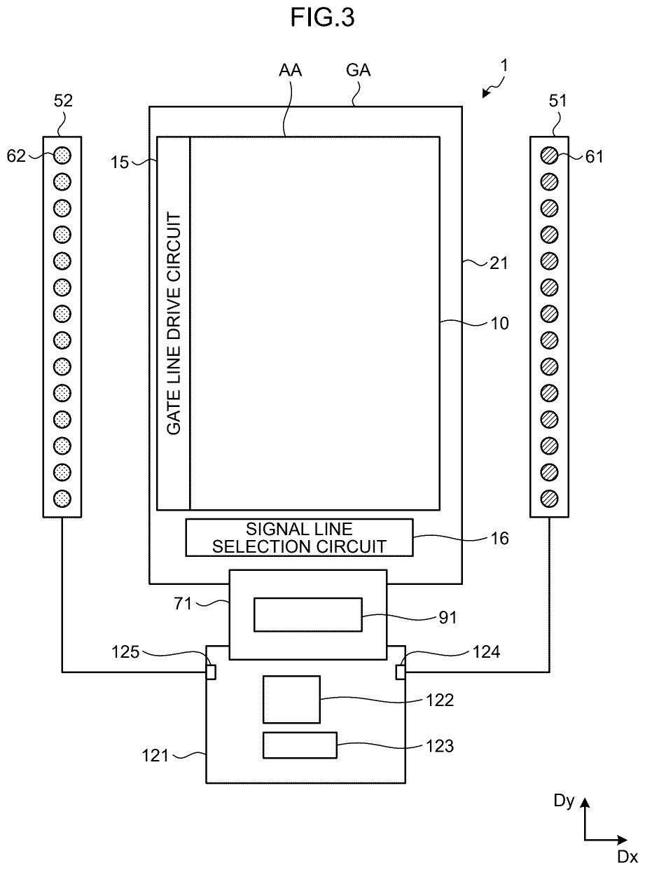

The following describes the sensor module 1 and the light source part 5 with reference to FIGS. 3 to 6.

FIG. 3 is a plan view illustrating an example of the sensor module 1, the light source part 5, and the configuration coupled to them. As illustrated in FIG. 3, the sensor module 1 includes a sensor base member 21, the sensor 10, a gate line drive circuit 15, a signal line selection circuit 16, the AFE 91, a control circuit 122, a power supply circuit 123, the first light source base member 51, the second light source base member 52, at least one first light source 61, and at least one second light source 62. While the embodiment describes a plurality of types of light sources (the first light source 61 and the second light source 62) as the light source, one type of light source may be provided.

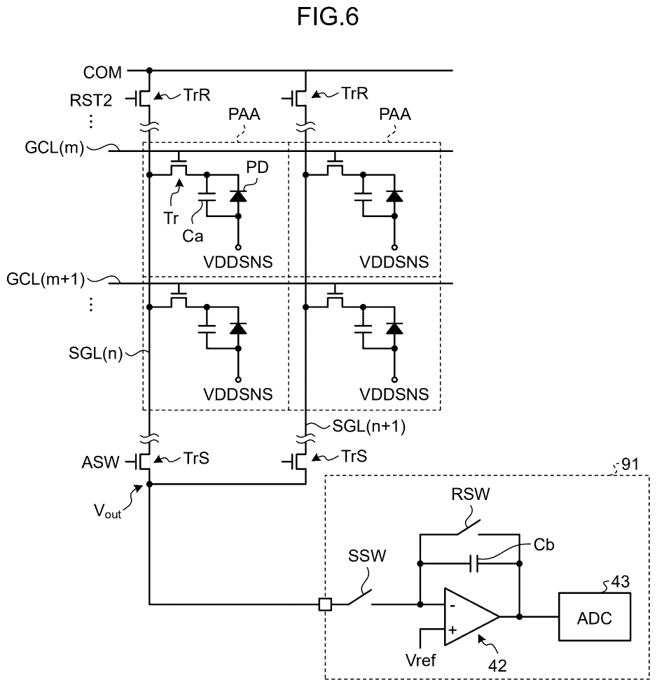

The sensor base member 21 is electrically coupled to a control substrate 121 via a flexible printed circuit board 71. The flexible printed circuit board 71 is provided with the AFE 91. The control substrate 121 is provided with the control circuit 122 and the power supply circuit 123. The control circuit 122 is a field-programmable gate array (FPGA), for example. The control circuit 122 supplies control signals to the sensor 10, the gate line drive circuit 15, and the signal line selection circuit 16 to control detection operations of the sensor 10. The control circuit 122 also supplies control signals to the first and the second light sources 61 and 62 to control lighting or non-lighting of the first and the second light sources 61 and 62. The power supply circuit 123 supplies voltage signals including, for example, a sensor power supply signal VDDSNS (refer to FIG. 6) to the sensor 10, the gate line drive circuit 15, and the signal line selection circuit 16. The power supply circuit 123 supplies a power supply voltage to the first and the second light sources 61 and 62 under the control of the AFE 91.

The sensor base member 21 has a detection area AA and a peripheral area GA. The detection area AA is an area provided with a plurality of photodiodes PD (refer to FIG. 6) included in the sensor 10. The peripheral area GA is an area between the outer perimeter of the detection area AA and the ends of the sensor base member 21 and is an area not overlapping the photodiodes PD.

The gate line drive circuit 15 and the signal line selection circuit 16 are provided in the peripheral area GA. Specifically, the gate line drive circuit 15 is provided in an area extending along a second direction Dy in the peripheral area GA. The signal line selection circuit 16 is provided in an area extending along a first direction Dx in the peripheral area GA and is provided between the sensor 10 and the AFE 91.

The first direction Dx is one direction in a plane parallel to the sensor base member 21. The second direction Dy is one direction in the plane parallel to the sensor base member 21 and is a direction orthogonal to the first direction Dx. The second direction Dy may non-orthogonally intersect the first direction Dx. A third direction Dz is a direction orthogonal to the first direction Dx and the second direction Dy and is a direction normal to the sensor base member 21.

The light sources 61 are provided on the first light source base member 51 and are arranged along the second direction Dy. The light sources 62 are provided on the second light source base member 52 and are arranged along the second direction Dy. The first light source base member 51 and the second light source base member 52 are electrically coupled to the control circuit 122 and the power supply circuit 123 through respective terminals 124 and 125 provided on the control substrate 121.

For example, inorganic light-emitting diodes (LEDs) or organic electroluminescent (EL) diodes (organic light-emitting diodes (OLEDs)) are used as the first and the second light sources 61 and 62. The first light sources 61 and the second light sources 62 emit first light L61 and second light L62, respectively, having different wavelengths. The first light L61 and the second light L62 have different emission maximum wavelengths. The emission maximum wavelength is the wavelength representing the maximum emission intensity in the emission spectrum indicating the relation between the wavelength and the emission intensity of the first light L61 and the second light L62. In the following description, when a numerical value of a wavelength is simply given, it represents an assumed emission maximum wavelength.

The first light L61 emitted from the first light sources 61 is mainly reflected on a surface of an object to be detected, such as a finger Fi, and enters the sensor 10. As a result, the sensor 10 can detect a fingerprint by detecting a shape of asperities on the surface of the finger Fi or the like. The second light L62 emitted from the second light sources 62 is mainly reflected in the finger Fi or the like, or transmitted through the finger Fi or the like, and enters the sensor 10. As a result, the sensor 10 can detect information on a living body in the finger Fi or the like. Examples of the information on the living body include, but are not limited to, pulse waves, pulsation, and a vascular image of the finger Fi or a palm.

For example, the first light L61 may have a wavelength of 520 nm to 600 nm, and the second light L62 may have a wavelength of 780 nm to 900 nm, for example, approximately 850 nm. In this case, the first light L61 is blue or green visible light, and the second light L62 is infrared light. The sensor 10 can detect the fingerprint based on the first light L61 emitted from the first light sources 61. The second light L62 emitted from the second light sources 62 is reflected in the object to be detected, such as the finger Fi, or transmitted through or absorbed by the finger Fi or the like, and is incident on the sensor 10. As a result, the sensor 10 can detect the pulse waves and the vascular image (vascular pattern) as the information on the living body in the finger Fi or the like.

Alternatively, the first light L61 may have a wavelength of 600 nm to 700 nm, for example, approximately 660 nm, and the second light L62 may have a wavelength of 780 nm to 900 nm, for example, approximately 850 nm. In this case, the sensor 10 can detect a blood oxygen saturation level in addition to the pulse waves, the pulsation, and the vascular image as the information on the living body based on the first light L61 emitted from the first light sources 61 and the second light L62 emitted from the second light sources 62. In this way, the detection device 100 includes the first and the second light sources 61 and 62, so that it performs the detection based on the first light L61 and the detection based on the second light L62, and thereby can detect the various types of information on the living body.

The arrangement of the first light sources 61 and the second light sources 62 illustrated in FIG. 3 is merely an example and can be changed as appropriate. For example, the first and the second light sources 61 and 62 may be arranged on each of the first and the second light source base members 51 and 52. In this case, a group including the first light sources 61 and a group including the second light sources 62 may be arranged in a second direction Dy, or the first and the second light sources 61 and 62 may be alternately arranged in the second direction Dy. The first and the second light sources 61 and 62 may be provided on one light source base member, or three or more light source base members. Alternatively, either the first light sources 61 or the second light sources 62 may be provided. However, when the pattern of the blood vessel Ve of the finger Fi is assumed to be acquired as in the embodiment, a light source that emits light including infrared light is preferably provided. The infrared light is more preferably near-infrared light, which is infrared light with a wavelength closer to visible light.

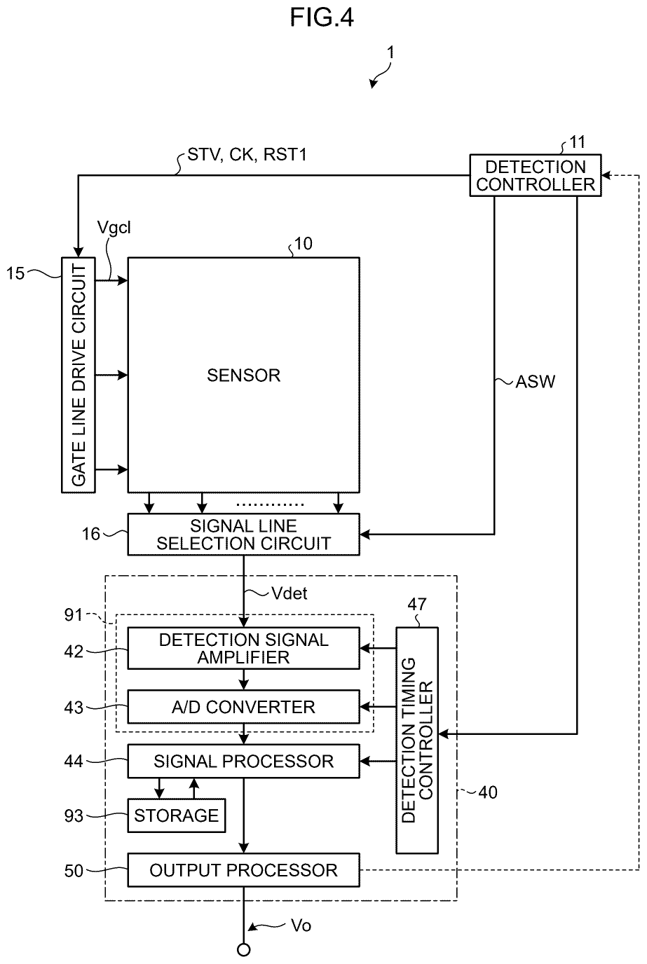

FIG. 4 is a block diagram illustrating a more detailed functional configuration example of the configuration illustrated in FIG. 3. As illustrated in FIG. 4, the sensor module 1 further includes a detection controller (detection control circuit) 11 and a detector (detection signal processing circuit) 40. The control circuit 122 includes one, some, or all functions of the detection controller 11. The control circuit 122 also includes one, some, or all functions of the detector 40 other than those of the AFE 91.

The sensor 10 is an optical sensor including the photodiodes PD serving as photoelectric conversion elements. Each of the photodiodes PD included in the sensor 10 outputs an electrical signal corresponding to light irradiating the photodiode PD to the signal line selection circuit 16. The signal line selection circuit 16 sequentially selects signal lines SGL based on a selection signal ASW from the detection controller 11. As a result, the electrical signal is output to the detector 40 as a detection signal Vdet. The sensor 10 performs detection in response to a gate drive signal Vgcl supplied from the gate line drive circuit 15.

The detection controller 11 is a circuit that supplies respective control signals to the gate line drive circuit 15, the signal line selection circuit 16, and the detector 40 to control operations of these components. The detection controller 11 supplies various control signals, such as a start signal STV, a clock signal CK, and a reset signal RST1, to the gate line drive circuit 15. The detection controller 11 also supplies various control signals, such as a selection signal ASW, to the signal line selection circuit 16. The detection controller 11 also supplies various control signals to the first and the second light sources 61 and 62 to control the lighting and the non-lighting of each group of the first and the second light sources 61 and 62.

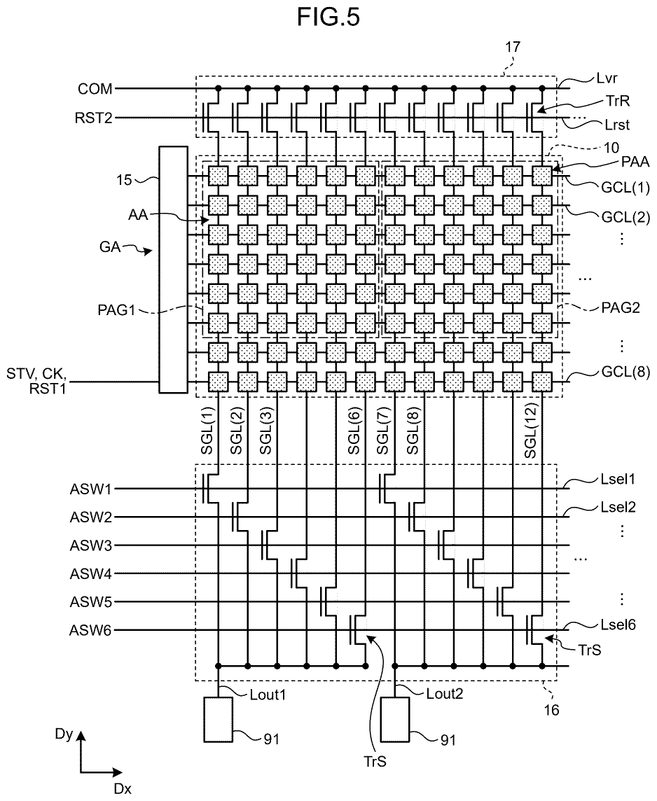

The gate line drive circuit 15 is a circuit that drives a plurality of gate lines GCL (refer to FIG. 5) based on various control signals. The gate line drive circuit 15 sequentially or simultaneously selects the gate lines GCL and supplies the gate drive signal Vgcl to the selected gate lines GCL. Through this operation, the gate line drive circuit 15 selects the photodiodes PD coupled to the gate lines GCL.

The signal line selection circuit 16 is a switch circuit that sequentially or simultaneously selects a plurality of signal lines SGL (refer to FIG. 5). The signal line selection circuit 16 is a multiplexer, for example. The signal line selection circuit 16 couples the selected signal lines SGL to the AFE 91 based on the selection signal ASW supplied from the detection controller 11. Through this operation, the signal line selection circuit 16 outputs the detection signals Vdet of the photodiodes PD to the detector 40.

The detector 40 includes the AFE 91, a signal processor 44, the storage 93, a detection timing controller 47, and an output processor 50. The detection timing controller 47 controls the AFE 91 and the signal processor 44 to operate in synchronization with each other based on the control signal supplied from the detection controller 11. As illustrated in FIG. 5 described later, the AFE 91 is circuitry including a plurality of AFE circuits each of which is provided for a plurality signal lines. In the following descriptions, each of the plurality of AFE included in the AFE 91 as entire circuitry is given the same reference sign “91” and is also referred to as the “AFE 91”.

The AFE 91 is a signal processing circuit having functions of, for example, a detection signal amplifier 42 and an analog-to-digital (A/D) converter 43. The detection signal amplifier 42 amplifies the detection signals Vdet. The A/D converter 43 converts analog signals output from the detection signal amplifier 42 into digital signals.

The signal processor 44 is a logic circuit that detects predetermined physical quantities received by the sensor 10 based on output signals of the AFE 91. The signal processor 44 can detect the asperities on the surface of the finger Fi or the palm based on the signals from the AFE 91 when the finger Fi is in contact with or in proximity to the detection area AA. The signal processor 44 can detect the information on the living body based on the signals from the AFE 91. Examples of the information on the living body include, but are not limited to, the vascular image, the pulse waves, the pulsation, and the blood oxygen saturation level of the finger Fi or the palm.

To obtain the blood oxygen saturation level of a human, for example, the wavelength of the first light L61 is set to 660 nm (the range is 500 nm to 700 nm), and the wavelength of the second light L62 is set to approximately 850 nm (the range is 800 nm to 930 nm). Since the amount of light absorbed changes depending on the amount of oxygen taken in by hemoglobin, the photodiode PD detects the amount of light obtained by subtracting the light absorbed by blood (hemoglobin) from the emitted first light L61 and second light L62. Most of the oxygen in blood is reversibly bound to hemoglobin in red blood cells, and a small portion is dissolved in blood plasma. More specifically, the value of the percentage of oxygen bound relative to the blood's total binding capacity is called the oxygen saturation level (SpO₂). With the two wavelengths of the first light L61 and the second light L62, the blood oxygen saturation level can be calculated from the amount obtained by subtracting the light absorbed by blood (hemoglobin) from the emitted light.

The signal processor 44 may acquire the detection signals Vdet (information on the living body) detected simultaneously by the photodiodes PD and perform processing of averaging them. In this case, the detector 40 can suppress measurement errors due to noise or relative misalignment between the sensor 10 and the object to be detected, such as the finger Fi, and perform stable detection.

The storage 93 temporarily stores therein signals calculated by the signal processor 44. The storage 93 may be, for example, a random-access memory (RAM) or a register circuit.

The output processor 50 functions as a processor that performs processing based on the output from the photodiodes PD. Specifically, the output processor 50 according to the embodiment outputs sensor output Vo including at least pulse wave data based on at least the detection signals Vdet acquired via the signal processor 44. In the embodiment, the signal processor 44 outputs data indicating the change (amplitude) of the output of the detection signal Vdet from each photodiode PD, which will be described later, and the output processor 50 determines which output is to be employed as the sensor output Vo. Both the output processing and the determination processing may be performed by the signal processor 44 or the output processor 50.

When a detection device for pulse waves or the like is worn on a human body, noise associated with breathing, changes in posture, or movement of the human body is also detected. For this reason, the signal processor 44 may be provided with a noise filter if necessary. The frequency component of the noise generated by breathing and changes in posture is, for example, equal to or lower than 1 Hz, which is sufficiently lower than the frequency component of the pulse waves. Therefore, the noise can be removed using a band-pass filter as the noise filter. The band-pass filter can be provided to the detection signal amplifier 42, for example. The frequency component of the noise caused by movement of the human body is, for example, on the order of several Hz to 100 Hz, which may overlap the frequency component of the pulse waves. The frequency in this case, however, is not constant but has fluctuations, so a noise filter that removes frequencies with fluctuation components is used. An example of the method for removing the frequencies with fluctuation components (first method for removing the fluctuation components) may use the property that the pulse waves have a time lag in the peak value depending on the measurement point on the human body. In other words, the pulse waves have a time lag depending on the measurement point on the human body, while noise generated by movement of the human body or the like does not have a time lag or has a smaller time lag than the pulse waves. Therefore, the pulse waves are measured at at least two different points, and if the peak values measured at different points fall within a predetermined time, the frequencies are removed as noise. Also in this case, it is possible that the waveform due to noise and the waveform due to the pulse waves may coincidentally overlap. In this case, however, the two waveforms overlap at only one of the different points, so the waveform due to noise and the waveform due to the pulse waves can be distinguished. This processing can be performed by the signal processor 44, for example. Another example of the method for removing the frequencies with fluctuation components (second method for removing the fluctuation components) is removing the frequency components with different phases by the signal processor 44. In this case, for example, a short-time Fourier transform may be performed to remove the fluctuation components, and then an inverse Fourier transform may be performed. While a commercial frequency power supply (50 Hz and 60 Hz) can also be a noise source, noise generated by the commercial frequency power supply does not have a time lag in the peak value measured at different points or has a smaller time lag than the pulse waves like the noise generated by movement of the human body or the like. Therefore, the noise can be removed by the same method as the first method for removing the fluctuation components described above. Alternatively, a shield may be provided on the surface of the detector opposite to the detection surface to remove the noise generated by the commercial frequency power supply.

The following describes a circuit configuration example of the sensor 10. FIG. 5 is a circuit diagram illustrating the sensor 10. FIG. 6 is a circuit diagram illustrating a plurality of partial detection areas. FIG. 6 also illustrates a circuit configuration of the AFE 91.

As illustrated in FIG. 5, the sensor 10 has a plurality of partial detection areas PAA arranged in a matrix having a row-column configuration. Each of the partial detection areas PAA is provided with the photodiode PD.

The gate lines GCL extend in the first direction Dx and are each coupled to the partial detection areas PAA arranged in the first direction Dx. A plurality of gate lines GCL(1), GCL(2), . . . , GCL(8) are arranged in the second direction Dy and are each coupled to the gate line drive circuit 15. In the following description, the gate lines GCL(1), GCL(2), . . . , GCL(8) will each be simply referred to as the gate line GCL when they need not be distinguished from one another. To facilitate understanding of the description, FIG. 5 illustrates eight gate lines GCL. However, this is merely an example, and M gate lines GCL may be arranged (where M is 8 or larger, e.g., M=256).

The signal lines SGL extend in the second direction Dy and are each coupled to the photodiodes PD of the partial detection areas PAA arranged in the second direction Dy. A plurality of signal lines SGL(1), SGL(2), . . . , SGL(12) are arranged in the first direction Dx and are each coupled to the signal line selection circuit 16 and a reset circuit 17. In the following description, the signal lines SGL(1), SGL(2), . . . , SGL(12) will each be simply referred to as the signal line SGL when they need not be distinguished from one another.

To facilitate understanding of the description, 12 signal lines SGL are illustrated. However, this is merely an example, and N signal lines SGL may be arranged (where N is 12 or larger, e.g., N=252). The resolution of the sensor is set to 508 dots per inch (dpi), for example, and the number of cells is 252×256. In FIG. 5, the sensor 10 is provided between the signal line selection circuit 16 and the reset circuit 17. The signal line selection circuit 16 and the reset circuit 17 are not limited to being provided in this way, and may be coupled to ends of the signal lines SGL on the same side. The substantial area of one sensor is, for example, substantially 50×50 μm², and the area of the detection area AA is, for example, 12.6×12.8 mm².

The gate line drive circuit 15 receives the various control signals such as the start signal STV, the clock signal CK, and the reset signal RST1 from the control circuit 122 (refer to FIG. 3). The gate line drive circuit 15 sequentially selects the gate lines GCL(1), GCL(2), . . . , GCL(8) in a time-division manner based on the various control signals. The gate line drive circuit 15 supplies the gate drive signal Vgcl to the selected one of the gate lines GCL. This operation supplies the gate drive signal Vgcl to a plurality of first switching elements Tr coupled to the gate line GCL, and thus selects the partial detection areas PAA arranged in the first direction Dx as detection targets.

The gate line drive circuit 15 may perform different driving for each of detection modes including the detection of the fingerprint and the detection of a plurality of different items of information on the living body (including, for example, the pulse waves, the pulsation, the vascular image, and the blood oxygen saturation level). For example, the gate line drive circuit 15 may collectively drive more than one of the gate lines GCL.

Specifically, the gate line drive circuit 15 may simultaneously select a predetermined number of the gate lines GCL from among the gate lines GCL(1), GCL(2), . . . , GCL(8) based on the control signals. For example, the gate line drive circuit 15 simultaneously selects six gate lines GCL(1) to GCL(6), and supplies thereto the gate drive signals Vgcl. The gate line drive circuit 15 supplies the gate drive signals Vgcl via the selected six gate lines GCL to the first switching elements Tr. This operation selects group areas PAG1 and PAG2 each including more than one of the partial detection areas PAA arranged in the first direction Dx and the second direction Dy as the detection targets. The gate line drive circuit 15 collectively drives the predetermined number of the gate lines GCL, and sequentially supplies the gate drive signals Vgcl to each unit of the predetermined number of the gate lines GCL. In the following description, the positions of the respective different group areas, such as the group areas PAG1 and PAG2, will each be referred to as the group area PAG when they are not particularly distinguished from one another.

The signal line selection circuit 16 includes a plurality of selection signal lines Lsel, a plurality of output signal lines Lout, and third switching elements TrS. The third switching elements TrS are provided correspondingly to the signal lines SGL. Six signal lines SGL(1), SGL(2), . . . , SGL(6) are coupled to a common output signal line Lout1. Six signal lines SGL(7), SGL(8), . . . , SGL(12) are coupled to a common output signal line Lout2. The output signal lines Lout1 and Lout2 are each coupled to the AFE 91.

The signal lines SGL(1), SGL(2), . . . , SGL(6) are grouped into a first signal line block, and the signal lines SGL(7), SGL(8), . . . , SGL(12) are grouped into a second signal line block. The selection signal lines Lsel are coupled to the gates of the respective third switching elements TrS included in one of the signal line blocks. One of the selection signal lines Lsel is coupled to the gates of the third switching elements TrS in the signal line blocks.

Specifically, selection signal lines Lsel1, Lsel2, . . . , Lsel6 are coupled to the third switching elements TrS corresponding to the signal lines SGL(1), SGL(2), . . . , SGL(6), respectively. The selection signal line Lsel1 is coupled to one of the third switching elements TrS corresponding to the signal line SGL(1) and one of the third switching elements TrS corresponding to the signal line SGL(7). The selection signal line Lsel2 is coupled to one of the third switching elements TrS corresponding to the signal line SGL(2) and one of the third switching elements TrS corresponding to the signal line SGL(8).

The control circuit 122 (refer to FIG. 3) sequentially supplies the selection signal ASW to the selection signal lines Lsel. This operation causes the signal line selection circuit 16 to operate the third switching elements TrS to sequentially select the signal lines SGL in one of the signal line blocks in a time-division manner. The signal line selection circuit 16 selects one of the signal lines SGL in each of the signal line blocks. Such a configuration can reduce the number of integrated circuits (ICs) including the AFE 91 or the number of terminals of the ICs in the sensor module 1.

The signal line selection circuit 16 may collectively couple more than one of the signal lines SGL to the AFE 91. Specifically, the control circuit 122 (refer to FIG. 3) simultaneously supplies the selection signals ASW to the selection signal lines Lsel. Thus, the signal line selection circuit 16 operates the third switching elements TrS to select the signal lines SGL (for example, six signal lines SGL) in one of the signal line blocks, and couples the signal lines SGL to the AFE 91. As a result, the signals detected in each group area PAG are output to the AFE 91. In this case, the signals from a plurality of partial detection areas PAA (photodiodes PD) are integrated in units of the group area PAG and output to the AFE 91.

The detection is performed for each group area PAG by the operations of the gate line drive circuit 15 and the signal line selection circuit 16. As a result, the strength of the detection signal Vdet obtained by a one-time detection operation increases, so that the sensor sensitivity can be improved. The time required for the detection can be reduced. As a result, the sensor module 1 can repeatedly perform the detection in a short time. Therefore, the sensor module 1 can improve the S/N ratio and accurately detect temporal changes in the information on the living body, such as the pulse waves.

As illustrated in FIG. 5, the reset circuit 17 includes a reference signal line Lvr, a reset signal line Lrst, and fourth switching elements TrR. The fourth switching elements TrR are provided correspondingly to the signal lines SGL. The reference signal line Lvr is coupled to either the sources or the drains of the fourth switching elements TrR. The reset signal line Lrst is coupled to the gates of the fourth switching elements TrR.

The control circuit 122 supplies a reset signal RST2 to the reset signal line Lrst. This operation turns on the fourth switching elements TrR to electrically couple the signal lines SGL to the reference signal line Lvr. The power supply circuit 123 supplies a reference signal COM to the reference signal line Lvr. This operation supplies the reference signal COM to a capacitive element Ca (refer to FIG. 6) included in each of the partial detection areas PAA.

As illustrated in FIG. 6, each of the partial detection areas PAA includes the photodiode PD, the capacitive element Ca, and a corresponding one of the first switching elements Tr. FIG. 6 illustrates two gate lines GCL(m) and GCL(m+1) arranged in the second direction Dy among the gate lines GCL. FIG. 6 also illustrates two signal lines SGL(n) and SGL(n+1) arranged in the first direction Dx among the signal lines SGL. The partial detection area PAA is an area surrounded by the gate lines GCL and the signal lines SGL. Each of the first switching elements Tr is provided correspondingly to the photodiode PD. The first switching element Tr is configured as a thin-film transistor, and in this example, configured as an n-channel metal oxide semiconductor (MOS) thin-film transistor (TFT).

The gates of the first switching elements Tr belonging to the partial detection areas PAA arranged in the first direction Dx are coupled to the gate line GCL. The sources of the first switching elements Tr belonging to the partial detection areas PAA arranged in the second direction Dy are coupled to the signal line SGL. The drain of the first switching element Tr is coupled to the cathode of the photodiode PD and the capacitive element Ca.

The anode of the photodiode PD is supplied with the sensor power supply signal VDDSNS from the power supply circuit 123. The signal line SGL and the capacitive element Ca are supplied with the reference signal COM serving as an initial potential of the signal line SGL and the capacitive element Ca from the power supply circuit 123.

When the partial detection area PAA is irradiated with light, a current corresponding to the amount of light flows through the photodiode PD. As a result, an electric charge is stored in the capacitive element Ca. When the first switching element Tr is turned on, a current corresponding to the electric charge stored in the capacitive element Ca flows through the signal line SGL. The signal line SGL is coupled to the AFE 91 through a corresponding one of the third switching elements TrS of the signal line selection circuit 16. Thus, the sensor module 1 can detect a signal corresponding to the amount of light irradiating the photodiode PD in each of the partial detection areas PAA or signals corresponding to the amounts of light irradiating the photodiodes PD in each of the group areas PAG.

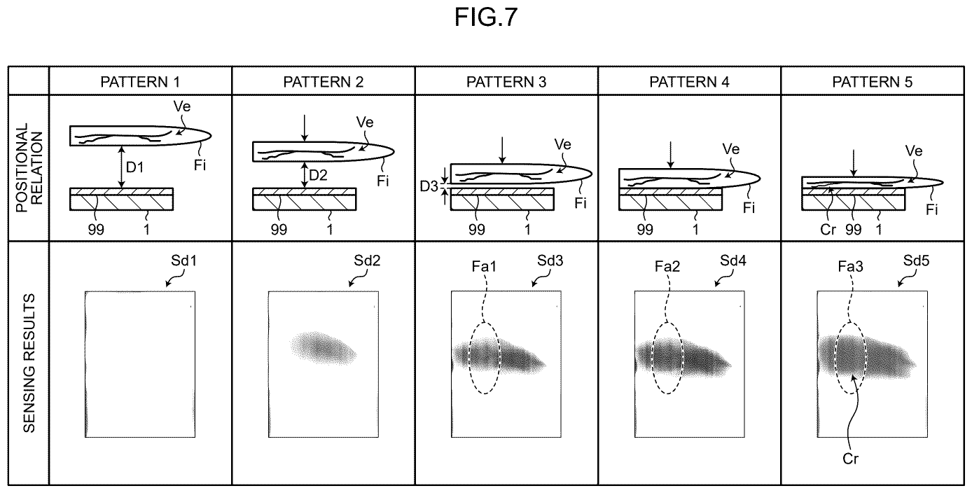

During a readout period Pdet (refer to FIG. 7), a switch SSW of the AFE 91 is turned on to couple the AFE 91 to the signal line SGL. The detection signal amplifier 42 of the AFE 91 converts a variation of a current supplied from the signal line SGL into a variation of a voltage, and amplifies the result. A reference potential (Vref) having a fixed potential is supplied to a non-inverting input part (+) of the detection signal amplifier 42, and the signal lines SGL are coupled to an inverting input terminal (−) of the detection signal amplifier 42. In the embodiment, the same signal as the reference signal COM is supplied as the reference potential (Vref) voltage. The detection signal amplifier 42 includes a capacitive element Cb and a reset switch RSW. During a reset period Prst (refer to FIG. 7), the reset switch RSW is turned on, and the electric charge of the capacitive element Cb is reset.

The AFE 91 illustrated in FIG. 1 functions at least as the A/D converter 43. The AFE 91 converts analog signals output from the sensor 10 into digital signals and outputs them to the controller 92. The AFE 91 may further function as the detection signal amplifier 42 as in the embodiment. The digital signal is a signal that can be interpreted by an arithmetic circuit included in the controller 92 and functioning as the signal processor 44. The AFE 91 outputs signals to control the lighting of the first light source 61 and the second light source 62 under the control of the controller 92. The controller 92 according to the embodiment to which the form described with reference to FIGS. 3 to 6 is applied includes the signal processor 44, the detection timing controller 47, and the output processor 50.

The controller 92 performs a plurality of processing steps to detect the vascular pattern of a human finger, such as determining that the finger Fi is detected and acquiring the pattern of the blood vessel Ve. The controller 92 makes a determination on the degree of force applied from the finger Fi to the sensor 10 and controls the notifier 94 based on the determination.

The AFE 91 and the controller 92 each include one or more circuits provided to implement the functions described above. The circuits may be a circuit in which a plurality of functions are integrated or circuits separately provided for the respective functions.

The notifier 94 includes a light source turned on or off based on the results of the determination on the degree of force applied from the finger Fi to the sensor 10. The light source is an LED or an OLED, for example, but is not limited thereto, and may have other specific configurations that function in the same way. The determination and the operations of the notifier 94 will be described later in greater detail.

The communicator 95 performs processing related to communications with external devices. The communicator 95 includes a circuit to function as a network interface controller (NIC) and performs processing related to communications with external devices according to a predetermined protocol. The communication path used for the communications may be a wired, wireless, or mixed wired and wireless circuit path, and may partially include a public communication network, such as the Internet.

While the controller 92, the storage 93, and the communicator 95 according to the embodiment are integrated into the control circuit 122, their specific implementation form is optionally determined. For example, some or all of the controller 92, the storage 93, and the communicator 95 may be provided as independent circuits.

Next, the degree of force applied from the finger Fi to the sensor 10 is described with reference to FIG. 7.

FIG. 7 is a schematic illustrating the relation between the degree of force applied from the finger Fi to the sensor 10 and the results of sensing by the sensor module 1. The sensing result herein indicates a light intensity pattern of the detection area obtained by the photodiodes PD disposed in the detection area individually detecting light. While the blood vessel Ve is assumed to be a vein, it may be an artery.

The sensor module 1 according to the embodiment can identify the blood vessel Ve in the finger Fi depending on the distance to the finger Fi even if the object to be detected (finger Fi) is not in contact with the louver 99. Specifically, as illustrated in “Pattern 1” in FIG. 7, when the distance between the louver 99 and the finger Fi is a distance D1, a sensing result Sd1 is obtained in which no shadow of light corresponding to the finger Fi, that is, no dark region in the sensing result is generated. As illustrated in “Pattern 1”, the louver 99 does not substantially create a shadow on the sensing result.

In the following description, when the term “dark region” is simply given, it refers to a dark region (low intensity region) generated in the sensing result by the shadow created by the light emitted from the light source part 5 toward the sensor 10 being blocked by the finger Fi. In other words, the white region around the dark region in the sensing result is a light region (high intensity region) generated by the light emitted from the light source part 5 toward the sensor 10 being detected substantially without any change. Therefore, the sensing result indicates a light intensity pattern in one of the following states: a state where only the light region is present, a state where only the dark region is present, and a state where the light region and the dark region are included.

As illustrated in “Pattern 2”, when the distance between the louver 99 and the finger Fi is a distance D2 shorter than the distance D1 in contrast to “Pattern 1” described above, a sensing result Sd2 is obtained in which a dark region corresponding to the finger Fi is generated. As illustrated in “Pattern 3”, when the distance between the louver 99 and the finger Fi is a distance D3 shorter than the distance D2, a sensing result Sd3 is obtained in which the dark region corresponding to the finger Fi and the pattern of the blood vessel Ve in the dark region are generated. The pattern of the blood vessel Ve in the sensing result Sd3 can be recognized in an area Fa1, for example.

As illustrated in “Pattern 4” in FIG. 7, when the finger Fi comes into contact with the louver 99, a sensing result Sd4 is obtained in which the dark region corresponding to the finger Fi is darker than that in the sensing result Sd3. The pattern of the blood vessel Ve in the sensing result Sd4 can be recognized in an area Fa2, for example.

By contrast, when the finger Fi is in contact with the louver 99 and the force pressing the finger Fi against the sensor 10 is too strong, such too strong force may flatten a portion of the blood vessel Ve in the finger Fi. The blood vessel Ve flattened in this manner is less likely to appear as an image in the sensing result. As illustrated in “Pattern 5” in FIG. 7, for example, the finger Fi is pressed hard against the louver 99, thereby causing a collapse Cr in the blood vessel Ve. The blood does not flow or is difficult to flow to the portion having the collapse Cr. As a result, the image of the blood vessel Ve is more difficult to recognize in an area Fa3 of a sensing result Sd5 than in the areas Fa1 and Fa2.

“Pattern 1” to “Pattern 5” in FIG. 7 can be considered as examples of the transition from a state where the finger Fi and the louver 99 are separated to a state where the finger Fi is pressed against the louver 99. In this case, the sensing results transition as the sensing results Sd1, Sd2, Sd3, Sd4, and Sd5, and the dark region in the sensing results gradually expands according to the transition.

Changes of the area occupied by the dark region in the sensing results may include the movement due to deviation in the position of the finger Fi or the like besides the expansion described above. It can be determined whether a change in the area occupied by the dark region in the sensing results corresponds to movement or expansion, based on a comparison of the sensing results before and after the transition.

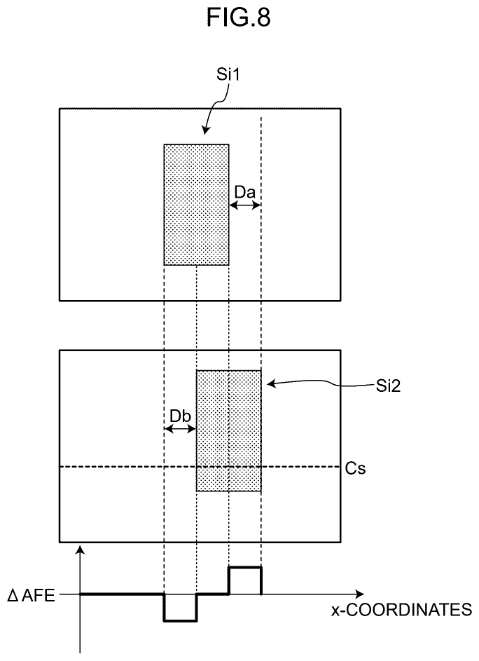

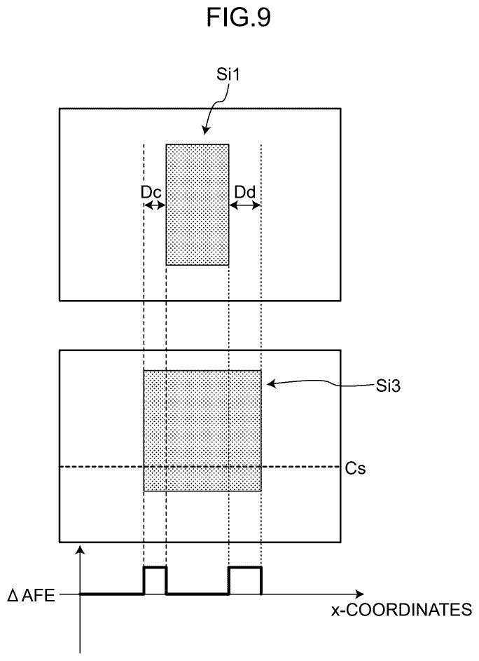

FIG. 8 is a schematic illustrating a case where a change in the area occupied by the dark region in the sensing results corresponds to movement. In FIG. 8 and FIG. 9, which will be described later, a dark region Si1 refers to a dark region included in the sensing result obtained by the sensing performed at the relatively earlier timing out of two sensing results obtained at different sensing timings. In FIG. 8, a dark region Si2 refers to a dark region included in the sensing result obtained by the sensing performed at the relatively later timing.

The dark region Si1 and the dark region Si2 illustrated in FIG. 8 are different in position in the first direction Dx. In each of FIG. 8 and FIG. 9, which will be described later, to illustrate a change in the area occupied by the dark region in the sensing results, the difference in whether the dark region is present in the first direction Dx is indicated by a graph. In the graph, the horizontal axis indicates the coordinates in the first direction Dx (x-coordinates), and the vertical axis indicates the output of the sensing results acquired by the AFE 91 from the sensor module 1. In the graph, a positive output in the vertical direction indicates a case where the dark region increases at a determination position Cs in the later-acquired sensing result, and a negative output in the vertical direction indicates a case where the dark region decreases in the later-acquired sensing result.

In comparison between the dark region Si1 and the dark region Si2 illustrated in FIG. 8, a negative output occurs in an area Db on one side in the x-coordinates, and a positive output occurs in an area Da on the other side in the x-coordinates. The area Db and the area Da are substantially identical. Therefore, when the output of the sensing results is viewed as a whole, the earlier-acquired sensing output including the dark region Si1 and the later-acquired sensing output including the dark region Si2 are substantially identical in the size of the area occupied by the dark region because the negative output and the positive output cancel each other out. Thus, if the size of the area occupied by the dark region does not change, the change in the dark region in the sensing results is considered to be due to movement of the finger Fi.

Therefore, assuming that the sensing result at one timing is the sensing result of “one frame”, if the dark region appearing in the sensing result of the relatively later frame is larger than the dark region appearing in the sensing result of the relatively earlier frame out of two or more frames at different timings, it can be considered that the force applied from the object to be detected (e.g., finger Fi), which generates the dark region, toward the detection area of the sensor 10 has increased between the timings. In particular, if the dark region includes the pattern of the blood vessel Ve, the object to be detected can be considered to be the finger Fi.

FIG. 9 is a schematic illustrating a case where a change in the area occupied by the dark region in the sensing results corresponds to expansion. In FIG. 9, a dark region Si3 refers to a dark region included in the sensing result obtained by the sensing performed at the relatively later timing.

In comparison between the dark region Si1 and the dark region Si3 illustrated in FIG. 9, a positive output occurs in an area Dc on one side in the x-coordinates and an area Dd on the other side in the x-coordinates. Therefore, when the output of the sensing results is viewed as a whole, the area occupied by the dark region is expanded in the later-acquired sensing output including the dark region Si3 compared with the earlier-acquired sensing output including the dark region Si1. Thus, if the size of the area occupied by the dark region changes, the change in the dark region in the sensing results is considered to be due to a change in the relative distance between the finger Fi and the sensor 10 (becoming closer) or an increase in the force from the finger Fi toward the sensor 10.

By contrast, if the dark region Si3 illustrated in FIG. 9 is the earlier-acquired sensing result and the dark region Si1 is the later-acquired sensing result, the change in the dark region in the sensing results, that is, the reduction in the area of the dark region is considered to be due to a change in the relative distance between the finger Fi and the sensor 10 (becoming farther away) or a decrease in the force from the finger Fi toward the sensor 10.

In the embodiment, it is possible to determine and notify the user that the force from the finger Fi toward the sensor 10 is too strong based on the occurrence of a collapse in the pattern of the blood vessel Ve in the dark region included in the sensing results, such as the collapse Cr described with reference to FIG. 7. To perform the determination and notification, the identification of an increase in force described with reference to FIGS. 8 and 9 may also be performed.

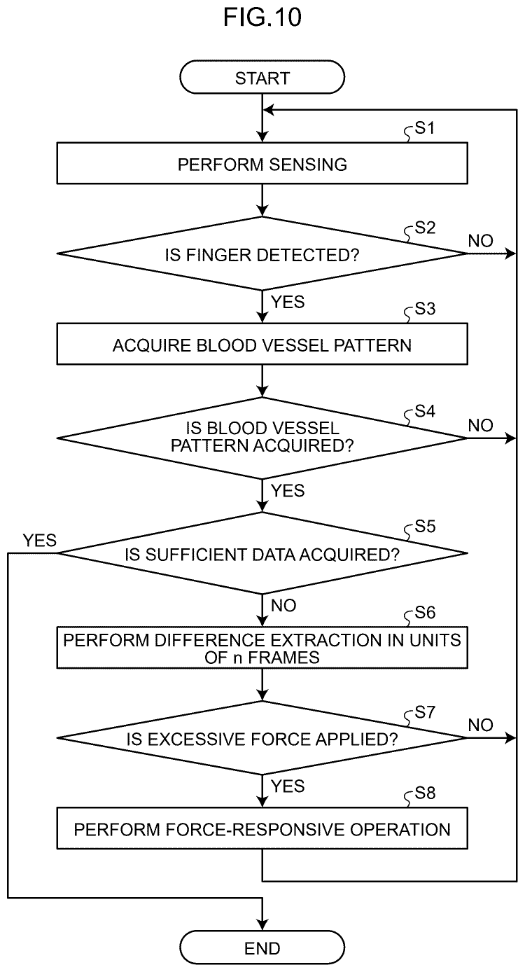

FIG. 10 is a flowchart illustrating the procedure performed by the detection device 100, including determination and notification that the force from the finger Fi toward the sensor 10 is too strong. First, sensing is performed (Step S1). Specifically, the photodiodes PD of the sensor module 1 operate to produce an output corresponding to the intensity of light emitted from the light source part 5 and detected by the photodiodes PD. The output is subjected to the processing by the AFE 91 and the controller 92 to become data that can be interpreted as the sensing results as described with reference to FIGS. 7 and 8.

The controller 92 determines whether a finger is detected by the sensing performed at Step S1 (Step S2). Specifically, the controller 92 performs determination processing to determine whether the sensing result obtained by the processing at Step S1 includes a dark region that can be determined to be generated by the finger Fi. The determination processing includes the following determinations: whether a dark region is generated; if a dark region is generated, whether the ratio of the dark region to the entire sensing result is such an appropriate ratio that the dark region can be considered to be generated by the finger Fi; and if a dark region is generated, whether the shape of the dark region is such a shape that the dark region can be considered to be generated by the finger Fi, for example. The specific contents of the determination processing can be appropriately modified. The appropriate ratio of the dark region to the entire sensing result is determined in advance based on prior tests or the like and held by the controller 92. For example, the appropriate ratio is in such a range that the dark region can be determined to be generated by the finger Fi. The determination based on the shape of the dark region is performed based on, for example, pattern matching with sample data of the dark region generated by the finger Fi that are prepared in advance. If the pattern matching is employed, the sample data is held in the controller 92 or a storage device that can be referenced from the controller 92 and is provided to the detection device 100.

If it is determined that no finger is detected at Step S2 (No at Step S2), the processing at Step S1 is performed again. In other words, the sensing is performed again. By contrast, if it is determined that a finger is detected at Step S2 (Yes at Step S2), the controller 92 performs the processing of acquiring the pattern of the blood vessel Ve from the sensing results obtained by the processing at Step S1 (Step S3). The processing of acquiring the pattern of the blood vessel Ve is performed based on, for example, the contrast between the blood vessel Ve and the area other than the blood vessel Ve in the dark region generated by the finger Fi, or pattern matching with the shape of the area determined to be likely to be the blood vessel Ve based on the contrast. The specific contents of the processing can be appropriately modified. If the pattern matching is employed, the sample data is held in the controller 92 or a storage device that can be referenced from the controller 92 and is provided to the detection device 100.

If the pattern of the blood vessel Ve is not acquired through the processing at Step S3 (No at Step S4), the processing at Step S1 is performed again. By contrast, if the pattern of the blood vessel Ve is acquired through the processing at Step S3 (Yes at Step S4), the controller 92 determines whether sufficient data according to the purpose for predetermined processing is acquired (Step S5). For example, assume that the predetermined processing is “personal authentication based on the blood vessel pattern”. In this case, one or more patterns of the blood vessel Ve that can be compared with blood vessel patterns prepared in advance simply need to be acquired by the processing at Step S3. Also assume that the predetermined processing is “measurement of the pulsation”. In this case, a plurality of sensing results simply need to be obtained at a predetermined cycle. As a result, the pulsation can be calculated from the relation between the pulses indicated by the patterns of the blood vessel Ve and the time length of the predetermined cycle. For the processing other than those described herein, the results of the processing at Step S5 also correspond to the specific contents of the predetermined processing.

If it is determined that sufficient data according to the purpose for the predetermined processing is acquired at Step S5 (Yes at Step S5), the process by the detection device 100 is terminated. By contrast, if it is determined that sufficient data according to the purpose for the predetermined processing is not acquired at Step S5 (No at Step S5), difference extraction is performed in units of n frames (Step S6). Specifically, the sensing results of the latest n times (n is a natural number of 2 or larger) are extracted out of the sensing results obtained by the processing at Step S1 that has already been performed after the start of the process and before the processing at Step S6. The extracted results are used in the processing at Step S7, which will be described later. If the number of times of the processing at Step S1 that has already been performed before the processing at Step S6 is smaller than n, all the sensing results are extracted to be used in the processing at Step S7.

The controller 92 determines whether excessive force from the finger Fi toward the sensor 10 is applied (Step S7). Specifically, the controller 92 compares a later-acquired sensing result with an earlier-acquired sensing result out of the sensing results extracted by the processing at Step S6. As a result of the comparison, if the pattern of the blood vessel Ve blurs or disappears in the later-acquired sensing result compared with the earlier-acquired sensing result, the controller 92 determines that excessive force is applied from the finger Fi toward the sensor 10 to such a degree that the collapse Cr described above is generated. At the determination processing, the controller 92 may determine that the cause of the disappearance of the pattern of the blood vessel Ve in the sensing result obtained by the later sensing is not due to movement of the finger Fi from the earlier-acquired sensing result using the same concept as that described with reference to FIG. 8. In other words, when movement of the finger Fi has occurred, the pattern of the blood vessel Ve in the finger Fi generated in the earlier-acquired sensing result is generated at the position corresponding to the finger Fi after the movement. In this case, the controller 92 determines that excessive force from the finger Fi toward the sensor 10 is not applied.

If it is determined that excessive force from the finger Fi toward the sensor 10 is applied at Step S7 (Yes at Step S7), the detection device 100 performs a force-responsive operation (Step S8). Specifically, the controller 92 lights up the notifier 94. Lighting up the notifier 94 enables notifying a person who can visually recognize the notifier 94, such as the user who is pressing the finger Fi toward the sensor 10, that excessive force from the finger Fi toward the sensor 10 is applied. In other words, the notifier 94 functions as a component lit up to notify the user that excessive force from the finger Fi toward the sensor 10 is applied.

After the processing at Step S8 or if it is determined that no excessive force from the finger Fi toward the sensor 10 is applied at Step S7 (No at Step S7), the processing at Step S1 is performed again.

Step S2 can be omitted. In other words, the processing at Step S3 may be automatically performed after the processing at Step S1. In this case, if the pattern of the blood vessel Ve is acquired at Step S4 (Yes at Step S4), the controller 92 may determine that the finger Fi is detected. In other words, the processing at Step S4 may also serve as the processing at Step S2.

The timing of the lighting of the notifier 94 is not necessarily limited to Step S8. For example, the notifier 94 may be lit up in a first lighting pattern if the pattern of the blood vessel Ve is acquired at Step S4 (Yes at Step S4) and in a second lighting pattern when the processing at Step S8 is performed. In this case, the first lighting pattern indicates that the pattern of the blood vessel Ve is normally acquired. The second lighting pattern indicates that the force from the finger Fi toward the sensor 10 is too strong. The first lighting pattern and the second lighting pattern are different lighting patterns, and those different lighting patterns can be set arbitrarily. For example, the color of the light source lit in the first lighting pattern may be different from that in the second lighting pattern. Alternatively, for the lighting patterns of a certain light source, the state of lighting in the first lighting pattern may be different from that in the second lighting pattern. Specifically, the light source remains lit in the first lighting pattern and repeatedly blinks in the second lighting pattern, for example.

As described above, a detection device (detection device 100) according to the embodiment includes a sensor (sensor 10) and an acquirer (controller 92). The sensor includes a plurality of optical sensor elements (photodiodes PD) arranged two-dimensionally. The acquirer acquires a pattern of a blood vessel (blood vessel Ve) of a human finger (finger Fi) that is included in a light intensity pattern of light detected by the sensor. The acquirer acquires information on force from the finger toward the sensor based on the light intensity pattern. Therefore, the embodiment enables detecting the force based on the light intensity pattern without requiring a dedicated force sensor for detecting the force.

As described with reference to FIGS. 7 and 8, for example, if a dark region in a later-acquired light intensity pattern out of a plurality of light intensity patterns acquired at different timings is larger than a dark region in an earlier-acquired light intensity pattern, the acquirer (controller 92) determines that the force from the finger (finger Fi) has increased during the acquisition of the plurality of light intensity patterns. Thus, the detection device can detect the force increase based on whether the dark region in the light intensity patterns expands.

As described with reference to FIG. 7, for example, if the pattern of the blood vessel included in an earlier-acquired light intensity pattern out of a plurality of light intensity patterns acquired at different timings blurs or disappears in a later-acquired light intensity pattern, the acquirer (controller 92) determines that the force from the finger (finger Fi) is too strong when the later-acquired light intensity pattern is acquired. Thus, the detection device can detect a state where the force is too strong based on the pattern of the blood vessel included in the light intensity patterns.

The detection device (detection device 100) also includes a notifier (notifier 94) that performs notification relating to the force from the finger (finger Fi) toward the sensor (sensor 10). With this configuration, the detection device can notify a person who can receive the notification by the notifier, such as a user who is pressing the finger (finger Fi) against the sensor, of information on the force.

The detection device (detection device 100) also includes a light source (second light source 62) that is provided at a position facing the surface provided with the optical sensor elements (photodiodes PD) and is configured to emit light including at least one of visible light and infrared light. With this configuration, the detection device facilitates producing the dark region generated by the finger (finger Fi) on the surface more reliably.

### Modifications

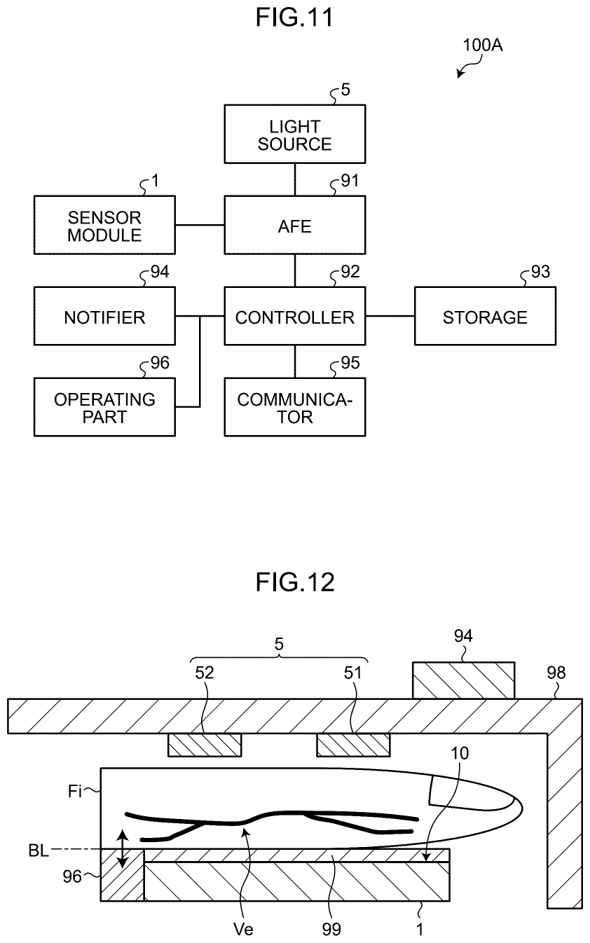

The following describes a modification of the embodiment with reference to FIGS. 11 and 12. In the description of the modification, the same components as those in the embodiment are not particularly described. They are denoted by the same reference numerals and the description thereof is omitted.

FIG. 11 is a block diagram illustrating a main configuration example of a detection device 100A according to the modification. The detection device 100A further includes an operating part 96 besides the components included in the detection device 100.

FIG. 12 is a schematic illustrating the positional relation between the sensor module 1, the light source part 5, the operating part 96, and the configuration around them in the detection device 100A, and the blood vessel Ve of the finger Fi serving as the object to be detected by the sensor module 1. The operating part 96 is switchable between a state where it protrudes toward the finger Fi and a state where it does not protrude toward the finger Fi with respect to the closest position of the finger Fi to the sensor 10. The “closest position of the finger Fi to the sensor 10” is, for example, a position BL of the surface of the louver 99 on the finger Fi side. In other words, the finger Fi is determined to be closest to the sensor 10 when the finger Fi is in contact with the surface of the louver 99 on the finger Fi side. When the louver 99 is not provided, the “closest position of the finger Fi to the sensor 10” is the surface provided with the photodiodes PD in the sensor 10, that is, the surface of the detection area.

When the force by the finger Fi is too high, the operating part 96 is driven to push up the finger Fi. The case where the force by the finger Fi is too high is represented by Pattern 5 described with reference to FIG. 7, for example. In such a case, the force applied by the finger Fi is considered to be too high (equal to or higher than a predetermined value). The operating part 96 includes, for example, a rotationally driven electric motor and an eccentric cam fixed to the output shaft of the electric motor and provided such that the outer diameter with respect to the center of rotation of the output shaft varies with the angle of rotation. In the operating part 96 according to the present example, the portion of the eccentric cam that is positioned on the side of the “closest position of the finger Fi to the sensor 10” changes depending on the angle of rotation of the rotating shaft. The operating part 96 is provided so as to produce both of the following angles of rotation of the rotating shaft: an angle of rotation of the rotating shaft at which a part of the outer peripheral surface of the eccentric cam protrudes from the “closest position of the finger Fi to the sensor 10” toward the finger Fi; and an angle of rotation of the rotating shaft at which the eccentric cam does not protrude from the “closest position of the finger Fi to the sensor 10” toward the finger Fi. The present example is merely an example of the specific configuration of the operating part 96, and the present modification is not limited thereto. For example, the operating part 96 may be an operating mechanism that is switchable between the states of protruding and not protruding from the “closest position of the finger Fi to the sensor 10” toward the finger Fi by linear motion of an actuator. The operating part 96 simply needs to be switchable between the states of protruding and not protruding toward the finger Fi with respect to the “closest position of the finger Fi to the sensor 10”, and its specific configuration is not particularly limited.

The operating part 96 operates to protrude from the “closest position of the finger Fi to the sensor 10” toward the finger Fi when the force applied from the finger Fi is determined to be too strong. In the modification, the controller 92 operates the operating part 96 such that it protrudes from the “closest position of the finger Fi to the sensor 10” toward the finger Fi at Step S8 described with reference to FIG. 10. As a result, the finger Fi receives biasing force in the direction away from the “closest position of the finger Fi to the sensor 10” from the operating part 96. The operation of the operating part 96 is intended to suggest that the user with the finger Fi reduce the force to the “closest position of the finger Fi to the sensor 10” through the sense of touch with the operating part 96. The user with the finger Fi has a feeling that the finger Fi is being pushed up from the “closest position of the finger Fi to the sensor 10” by the operating part 96, thereby readily noticing that the force applied from the finger Fi toward the sensor 10 is too strong.

In the modification, if it is determined that no excessive force is applied from the finger Fi toward the sensor 10, in the processing at Step S7 performed after the processing at Step S8 has been performed one or more times (No at Step S7), the controller 92 operates the operating part 96 such that it does not protrude from the “closest position of the finger Fi to the sensor 10” toward the finger Fi. Thus, the user with the finger Fi readily notices that the state where the force applied from the finger Fi toward the sensor 10 is too strong has been resolved.

In the modification, not only the operation control on the operating part 96 but also the lighting control on the notifier 94 may be performed as in the embodiment. The modification is the same as the embodiment, except in the matters noted above.

As described above, the detection device (detection device 100A) according to the modification includes an operating part (operating part 96) provided adjacent to the sensor (sensor 10) and switchable between a state of protruding toward the finger and a state of not protruding toward the finger depending on the force from the finger toward the sensor with respect to the closest position (position BL) of the finger (finger Fi) to the sensor. With this configuration, the detection device can perform output according to the force via the operating part.

The operating part (operating part 96) protrudes toward the finger when the force applied from the finger (finger Fi) is determined to be too strong. Thus, the detection device can suggest to the user who presses the finger (finger Fi) against the sensor (sensor 10) that the force is too strong through the sense of touch generated on the finger by the operating part.

The configuration that functions as a notifier is not limited to the notifier 94 or the operating part 96, and its specific configuration can be appropriately modified. For example, the notifier may be a sound output device that notifies the user that the force is too strong by sound. The sound output device, for example, includes a speaker, an amplifier, a storage device that stores therein sound data, and other components. Alternatively, the notifier may be a display device that performs notification by outputting images, such as images including character information. The display device includes a display, a display driver circuit, a storage device that stores therein image data to be output on the display, and other components.

Other operational advantages accruing from the aspects described in the present embodiment that are obvious from the description herein, or that are conceivable as appropriate by those skilled in the art will naturally be understood as accruing from the present disclosure.

What is claimed is:

1. A detection device comprising:
a sensor comprising a plurality of optical sensor elements arranged two-dimensionally; and
an acquirer configured to acquire a pattern of a blood vessel of a human finger that is included in a light intensity pattern of light detected by the sensor, wherein
the acquirer is configured to acquire information on force from the finger toward the sensor based on the light intensity pattern.

2. The detection device according to claim 1, wherein the acquirer is configured to determine, when a low intensity region in a later-acquired light intensity pattern out of a plurality of the light intensity patterns acquired at different timings is larger than a low intensity region in an earlier-acquired light intensity pattern, that the force from the finger has increased during the acquisition of the plurality of light intensity patterns.

3. The detection device according to claim 1, wherein the acquirer is configured to determine, when the pattern of the blood vessel included in an earlier-acquired light intensity pattern out of a plurality of the light intensity patterns acquired at different timings blurs or disappears in a later-acquired light intensity pattern, that the force from the finger is too strong when the later-acquired light intensity pattern is acquired.

4. The detection device according to claim 1, further comprising a notifier configured to perform notification relating to the force from the finger toward the sensor.

5. The detection device according to claim 1, further comprising a light source that is provided at a position facing a surface provided with the optical sensor elements and is configured to emit light including at least one of visible light or infrared light.

6. The detection device according to claim 3, further comprising an operating part provided adjacent to the sensor and switchable between a state of protruding toward the finger and a state of not protruding toward the finger depending on the force from the finger toward the sensor with respect to the closest position of the finger to the sensor.

7. The detection device according to claim 6, wherein the operating part protrudes toward the finger when the force applied from the finger is determined to be too strong.
